(19) United States
(12) Patent Application Publication
(10) Pub. No.: US 2014/0296288 A1
(43) Pub. Date: Oct. 2, 2014

(54) IMINO-SUGAR C-GLYCOSIDES, PREPARATION AND USE THEREOF

(71) Applicants: Matthieu Sollogoub, Paris (FR); Yves Bleriot, Poitiers (FR); Giuseppe Prencipe, Paris (FR); Nicolas Auberger, Poitiers (FR)

(72) Inventors: Matthieu Sollogoub, Paris (FR); Yves Bleriot, Poitiers (FR); Giuseppe Prencipe, Paris (FR); Nicolas Auberger, Poitiers (FR)

(73) Assignees: UNIVERSITE PIERRE ET MARIE CURIE (PARIS 6), PARIS (FR); DORPHAN S.A., LAUSANNE (CH); CENTRE NATIONAL DE LA RECHERCHE SCIENTIFIQUE, PARIS (FR)

(21) Appl. No.: 14/233,713

(22) PCT Filed: Jul. 19, 2012
(86) PCT No.: PCT/EP2012/064212

§ 371 Date: Jun. 3, 2014

(30) Foreign Application Priority Data
Jul. 20, 2011 (EP) 11305942.2

(51) Int. Cl. C07D 211/56

(52) U.S. Cl. CPC C07D 211/56
USPC 514/315; 546/242; 435/184

(57) ABSTRACT

The present disclosure relates to iminosugar derivatives and processes for the preparation thereof. The disclosed compounds have glycosidase inhibiting properties, and are useful in the treatment of various diseases, such as type 2 diabetes, neurodegenerative diseases or lysosomal storage disorders. The present disclosure also relates to pharmaceutical compositions containing the disclosed compounds and to their use as biochemical tools.

### CROSS-REFERENCE TO RELATED APPLICATIONS

This application is the U.S. National Stage under 37 U.S.C. §371 of International Application No. PCT/EP2012/064212, filed Jul. 19, 2012, which claims priority to European Application No. 11305942.2, filed Jul. 20, 2011. The International Application published on Jan. 24, 2013 as WO 2013/011098, and republished on May 30, 2013. All of the above applications are incorporated by reference in their entireties.

### FIELD OF THE INVENTION

The present disclosure relates to iminosugar derivatives and processes for the preparation thereof. The disclosed compounds have glycosidase inhibiting properties, and are useful in the treatment of various diseases, such as type 2 diabetes, neurodegenerative diseases or lysosomal storage disorders. The present disclosure also relates to pharmaceutical compositions containing the disclosed compounds and to their use as biochemical tools.

### BACKGROUND OF THE INVENTION

The search for selective and effective inhibitors of oligosaccharide processing enzymes has been the object of intensive research over the last 20 years in the synthesis of stereochemically well-defined polyhydroxylated piperidines. The search for promising glycosidase inhibitors led to the discovery of homoiminosugars or homoglyconojirimycins where the pseudoanomeric OH group of unstable nojirimycin has been homologated to yield compounds which are stable towards chemical and enzymatic degradation while retaining the powerful biological activity of the parent iminosugar. Furthermore, homoiminosugars display a pseudoanomeric substituent of defined stereochemistry which can strongly interact with the aglycon-binding site of the glycosidase and is expected to further increase the selectivity towards these enzymes. Due to their higher selectivity and potency as glycosidase inhibitors, the homoiminosugars are gaining their own independent identity. Homoiminosugar mimics of all the key glycosides involved in the maturation of glycoproteins including mannose A, glucose B, galactose C and fucose D have been synthesized in the past in both anomeric configurations (see structures below). 2-Acetamido-1,2-dideoxy-nojirimycin E and 2-acetamido-2-deoxy-nojirimycin F (see structures below) have been synthesized and extensively studied, demonstrating high biological potential of these GlcNAc piperidine mimics (GlcNAc stands for N-acetylglucosamine) as probes or inhibitors of glycosidases responsible for the cleavage of N-acetyl-D-glucosamine in glycoconjugates.

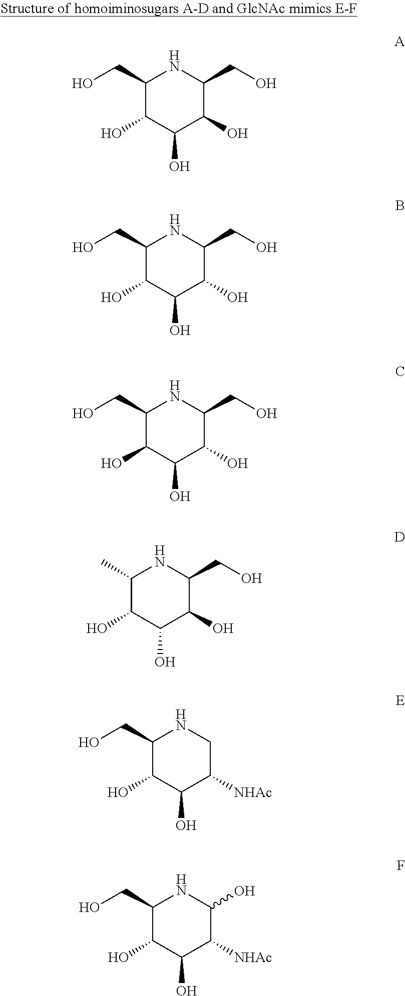

There is still a need for the discovery of new and improved glycosidase inhibitors, with potentially an improved toxicity profile.

### SUMMARY OF THE INVENTION

The inventors have now developed iminosugar-C-glycosides derived from N-acetyl-D-glucosamine and processes for the preparation thereof.

The disclosed compounds have glycosidase inhibiting properties, in particular N-acetyl hexosaminidase inhibiting properties, and are therefore useful in the treatment of various diseases, such as type-2 diabetes, neurodegenerative diseases or lysosomal storage disorders. Glycosidase cell-based assays using fibroblasts from patients suffering from Sanfilippo syndrome (a lysosomal storage disease) are particularly promising for development of one of the disclosed compound for treatment of this disorder.

The present disclosure also relates to pharmaceutical compositions containing the disclosed compounds and to their use as biochemical tools, in particular to assess glycosidase mediated enzymatic hydrolysis reaction pathways and mechanisms.

### BRIEF DESCRIPTION OF THE DRAWINGS

FIG. 1: Enzyme NAGLU activity (FU/μg protein) obtained in 3 different MPS IIIB fibroblast cell lines (GM01426—FIG. 1(a), GM00737—FIG. 1(b), and GM02931—FIG. 1(c)) at various concentrations of compound 1 of the invention and compound (B) (0-10 μM).

### DETAILED DESCRIPTION

Accordingly, and in a first aspect of the invention, it is herein disclosed a compound of general formula (I):

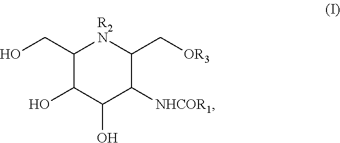

wherein:

R1 represents an alkyl (C1-C10) group, an alkenyl (C2-C10) group, an alkynyl (C2-C10) group, a cycloalkyl (C3-C10) group, a heterocycle (C3-C18) group, an aryl (C6-C18) group, or an arylalkyl group;

R2 represents a hydrogen atom, an alkyl (C1-C10) group, an alkenyl (C2-C10) group, an alkynyl (C2-C10) group, a cycloalkyl (C3-C10) group, a heterocycle (C3-C18) group, an aryl (C6-C18) group, or an arylalkyl group;

R3 represents a hydrogen atom, an alkyl (C1-C10) group, an alkenyl (C2-C10) group, an alkynyl (C2-C10) group, a cycloalkyl (C3-C10) group, a heterocycle (C3-C18) group, an aryl (C6-C18) group, or an arylalkyl group;

any geometrical or optical isomer thereof.

According to the invention, the hydroxyl and/or amino groups of compounds of formula (I) may be independently further protected with an appropriate protecting group. The compounds of formula (I) also include compounds of formula (I) presenting one or more amino or alcohol protecting groups.

Alcohol protecting groups are well known in the art. One can cite for instance methyl, benzyl, acetyl, benzoyl, β-methoxyethoxymethyl ether, methoxymethyl ether, or p-methoxybenzyl ether.

Amino-protecting groups are well known in the art. One can cite for instance tert-butyloxycarbonyl, carbobenzyloxy, p-methoxybenzyl carbonyl, 9-fluorenylmethyloxycarbonyl, benzyl, acetyl, or benzoyl.

According to the invention, the groups identified above may be substituted or unsubstituted. In general, the term “substituted” refers to a functional group, as defined below, in which one or more bonds to a hydrogen atom are replaced by a bond to a non-hydrogen atom. Substituted groups also include groups, in which one or more bonds to a hydrogen atom are replaced by one or more bonds, including double or triple bonds, to a heteroatom. In some embodiments, substituted groups have 1, 2, 3, 4, 5, or 6 substituents. Examples of substituent groups include, but are not limited to, halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls (oxo); carboxyls; esters; ethers; urethanes; oximes; hydroxylamines; alkoxyamines; thiols; sulfides such as alkyl, alkenyl, alkynyl, aryl, aralkyl, heterocyclyl and heterocyclylalkyl sulfide groups; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitriles; alkyl, alkenyl, alkynyl, aryl, aralkyl, heteroaryl, heterocyclyl, heterocyclylalkyl and cycloalkyl groups.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups may also be substituted with alkoxy, alkyl, alkenyl, and alkynyl groups as defined below.

According to the invention, the term “alkyl (C1-C10)” designates a saturated hydrocarbonated group, straight or branched, having from 1 to 10, preferably from 1 to 6, carbon atoms. Examples of straight chain alkyl groups include, but are not limited to, those with from 1 to 10 carbon atoms such as methyl, ethyl, n-propyl, n-butyl, n-pentyl, n-hexyl groups, n-heptyl, n-octyl, n-nonyl and n-decyl groups. Examples of branched alkyl groups include, but are not limited to, isopropyl, iso-butyl, sec-butyl, tert-butyl, isopentyl, and 2,2-dimethylpropyl groups. Alkyl groups may be substituted or unsubstituted. Representative substituted alkyl groups may be substituted one or more times with any of the groups listed above, for example, amino, oxo, hydroxy, cyano, carboxy, nitro, thio, alkoxy, F, Cl, Br, I, cycloalkyl, aryl, heterocyclyl and heteroaryl groups.

Cycloalkyl (C3-C10) groups are cyclic alkyl groups having from 3 to 10 carbon atoms such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms ranges from 3 to 7, in particular is 3, 4, 5, 6, or 7. Cycloalkyl groups further include mono-, bicyclic and polycyclic ring systems, such as, for example bridged cycloalkyl groups as described below, such as, but not limited to, adamantyl, and fused rings, such as, but not limited to, decalinyl, and the like. Cycloalkyl groups may be substituted or unsubstituted. Cycloalkyl groups may be substituted one or more times with non-hydrogen groups as defined above (substituents). However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with any of the groups listed above, for example, methyl, amino, hydroxy, cyano, carboxy, nitro, thio, alkoxy, and F, Cl, Br, I groups.

Alkenyl (C2-C10) groups include straight and branched chain alkyl and cycloalkyl groups as defined above, except that at least one double bond exists between two carbon atoms. Thus, alkenyl groups have from 2 to 10 carbon atoms. Examples include, but are not limited to, vinyl, CH═CH(CH3), CH═C(CH3)2, C(CH3)=CH2, C(CH3)=CH(CH3), C(CH2CH3)=CH2, cyclohexenyl, cyclopentenyl, cyclohexadienyl, butadienyl, pentadienyl, and hexadienyl, among others. Alkenyl groups may be substituted or unsubstituted.

Alkynyl (C2-C10) groups include straight and branched chain alkyl groups, except that at least one triple bond exists between two carbon atoms. Thus, alkynyl groups have from 2 to 10 carbon atoms. Examples include, but are not limited to, 1-ethynyl, 1-propynyl, 2-propynyl, 1-butynyl, 2-butynyl, 1-pentynyl or 2-pentynyl radical, among others. Alkynyl groups may be substituted or unsubstituted.

The term alkyloxy or alkoxy refers to an alkyl chain linked to the molecule by means of an oxygen atom (ether linkage). The alkyl chain corresponds to the definition given above. As an example, one can cite the methoxy, ethoxy, n-propyloxy, isopropyloxy, n-butoxy, iso-butoxy, tert-butoxy, sec-butoxy, hexyloxy radicals. The same definition applies to alkenoxy, alkynoxy, aryloxy, aralkyloxy, heterocyclyloxy, and heterocyclylalkoxy groups, where the chains are linked to the molecule by means of an oxygen atom.

Aryl groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. The phenyl groups, substituted or not, are particularly preferred. In some embodiments, aryl groups contain 6-14 carbons, and in others from 6 to 12 or even 6-10 carbon atoms in the ring portions of the groups. The phrase “aryl groups” includes groups containing fused rings, such as fused aromatic-aliphatic ring systems (e.g., indanyl, tetrahydronaphthyl, and the like). Aryl groups may be substituted or unsubstituted. Groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with groups such as those listed above.

Heterocyclyl groups are non-aromatic ring groups containing 3 or more ring members, of which one or more is a heteroatom such as, but not limited to, N, O, and S. In some embodiments, the heterocyclyl group contains 1, 2, 3, or 4 heteroatoms. In some embodiments, heterocyclyl groups include 3 to 6, 10, 12, or 15 ring atoms. Heterocyclyl groups encompass unsaturated, partially saturated and saturated ring systems, such as, for example, imidazolinyl and imidazolidinyl groups. The phrase “heterocyclyl group” includes fused ring species including those comprising fused aromatic and non-aromatic groups, such as, for example, 2,3-dihydrobenzo[1,4]dioxinyl. The phrase also includes bridged polycyclic ring systems containing a heteroatom such as, but not limited to, quinuclidyl. Heterocyclyl groups may have other groups, such as alkyl, oxo or halo groups, bonded to one of the ring members. These are referred to as “substituted heterocyclyl groups.” Heterocyclyl groups may be substituted or unsubstituted. Heterocyclyl groups include, but are not limited to, pyrrolidinyl, pyrrolinyl, imidazolinyl, imidazolidinyl, piperidinyl, piperazinyl, morpholinyl, pyrazolidinyl, tetrahydropyranyl, thiomorpholinyl, pyranyl, tetrahydrofuranyl, dihydrobenzofuranyl, dihydroindolyl, azabenzimidazolyl, benzothiadiazolyl, imidazopyridinyl, thianaphthalenyl, xanthinyl, guaninyl, tetrahydroquinolinyl, and 2,3-dihydrobenzo[1,4]dioxinyl. Representative substituted heterocyclyl groups may be mono-substituted or substituted more than once, such as, but not limited to, triazolyl, pyridinyl or morpholinyl groups, which are 1-, 2-, 3-, 4-, 5-, or 6-substituted, or disubstituted with various groups as defined above, including, but not limited to, alkyl, oxo, carbonyl, amino, alkoxy, cyano, and/or halo.

Heteroaryl groups are cyclic aromatic hydrocarbons that contain one or more heteroatoms such as, but not limited to, N, O, and S. In some embodiments, the heteroaryl group contains 1, 2, 3, or 4 heteroatoms. In some embodiments, heteroaryl groups include 3 to 6, 10, 12, or 15 ring atoms. The phrase “heteroaryl group” includes fused ring species, such as benzotriazolyl and benzo[1,3]dioxolyl. Heteroaryl groups may have other groups, such as alkyl, oxo or halo groups, bonded to one of the ring members. These are referred to as “substituted heteroaryl groups.” Heteroaryl groups may be substituted or unsubstituted. They can also be partially aromatic, such as tetrazolyl. Heteroaryl groups include, but are not limited to, imidazolyl, pyrrolyl, pyrazolyl, triazolyl, tetrazolyl, furanyl, oxazolyl, isoxazolyl, thiazolyl, pyridinyl, pyrimidinyl, pyridazinyl, pyrazinyl, thiophenyl, benzothiophenyl, benzofuranyl, indolyl, azaindolyl, indazolyl, benzimidazolyl, benzoxazolyl, benzothiazolyl, isoxazolopyridinyl, purinyl, adeninyl, quinolinyl, isoquinolinyl, quinoxalinyl, quinazolinyl, benzotriazolyl, and benzo[1,3]dioxolyl groups.

The term arylalkyl group denotes a radical of the alkyl type as defined above substituted by an aryl group as defined above. The benzyl and phenethyl groups are particularly preferred.

The compounds discussed herein also encompass their stereoisomers (diastereoisomers, enantiomers), pure or mixed, racemic mixtures, geometrical isomers, tautomers, salts, hydrates, solvates, solid forms as well as their mixtures. Some compounds according to the invention and their salts could be stable in several solid forms. The present invention includes all the solid forms of the compounds according to the invention which includes amorphous, polymorphous, mono- and polycrystalline forms.

The compounds according to the invention can exist in non-solvated or solvated form, for example with pharmaceutically acceptable solvents such as water (hydrates) or ethanol.

More specifically, the present invention relates to a compound of the following formula:

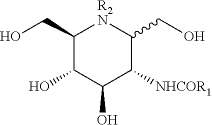

wherein R1 and R2 are as defined above.

More specifically, the compound is one of the following formulae:

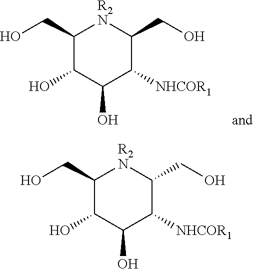

wherein R1 and R2 are as defined above.

Preferably, R2 is a hydrogen atom, an alkyl (C1-C10) group, or a cycloalkyl (C3-C10) group. Preferably, R1 represents an alkyl (C1-C6) group, preferably methyl or trifluoromethyl, a cycloalkyl (C3-C10) group, preferably adamantyl, or a heterocycle (C3-C18) group, and preferably R2 is a hydrogen atom, an alkyl (C1-C10) group, or a cycloalkyl (C3-C10) group. According to a specific embodiment, R2 is a hydrogen atom and/or R1 is a methyl group. According to another embodiment, R2 is an alkyl (C8-C10) group, optionally substituted with at least one heteroaryl and/or heterocycle and/or cycloalkyl (C3-C10) group and/or alkoxy group.

In a specific embodiment, R2 is an alkyl (C1-C10) group, substituted by one heteroaryl group (preferably triazolyl), said group being substituted by an alkyl group interrupted by an heteroatom, preferably an oxygen atom, and said alkyl group being substituted by a cycloalkyl (C3-C10) group, preferably an adamantyl group.

Compounds of formula (I) can be used for biological or therapeutical purposes or for further synthesis, in particular for use as a scaffold for covalent binding to another group or molecule, such as an organophosphate, a phosphoric acid group, an amino acid, a carbohydrate, a protein, or a peptide. The —CH2OR3 group (or more specifically —CH2OH group) of the compound of formula (I) can be modified and replaced by a —CH2R group (where R is as defined below).

According to another embodiment, the invention relates to a compound of general formula (II):

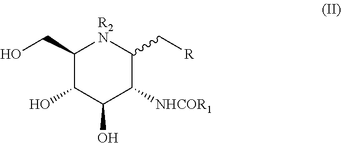

wherein R1 and R2 are as defined above, including the described specific embodiments, and R represents an halogen atom, such as fluoro, or represents a group containing an aliphatic and/or aromatic group, for example alkoxy (e.g. methyloxy (—OMe), ethyloxy (—OEt)), halogenoalkoxy (e.g., —OEtBr), nitrophenoxy, organophosphate, phosphoric acid group, amino acid, peptide, protein, carbohydrate or derivative thereof.

More specifically, the compound of formula (II) is one of the following formulae:

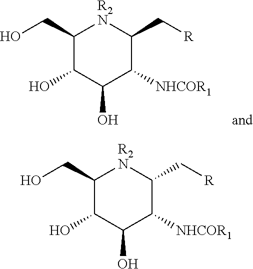

wherein R, R1 and R2 are as defined above.

Illustrative compounds particularly useful in the invention are the following.

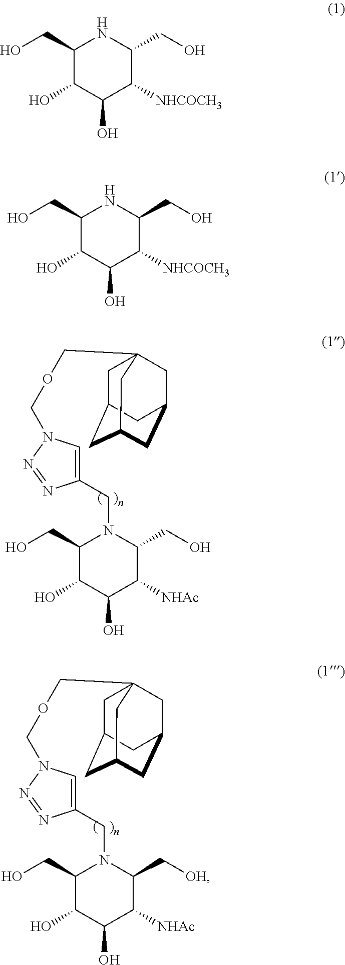

wherein n is an integer from 1 to 10.

Preferably, n is 8, 9 or 10.

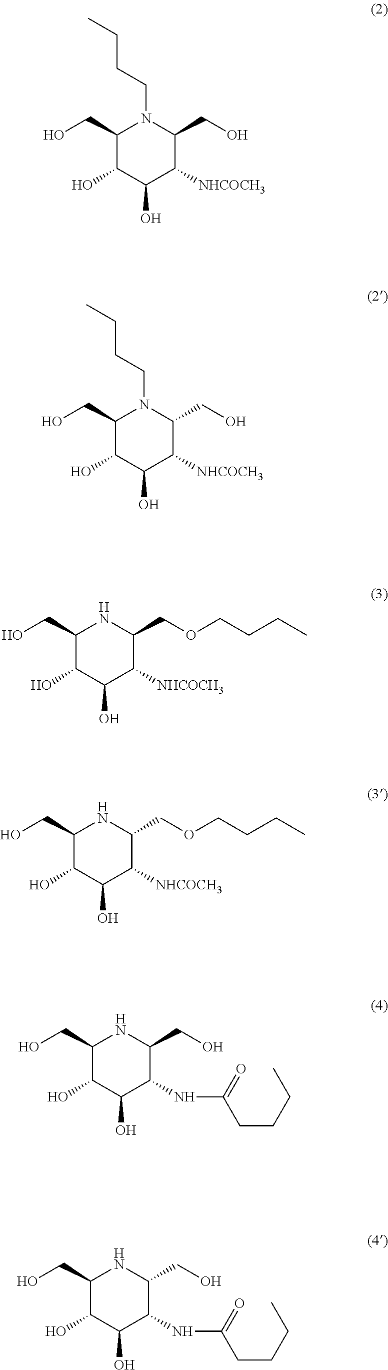

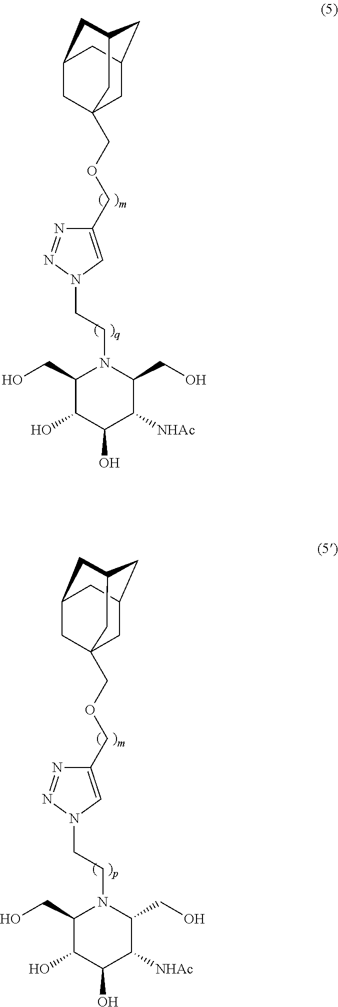

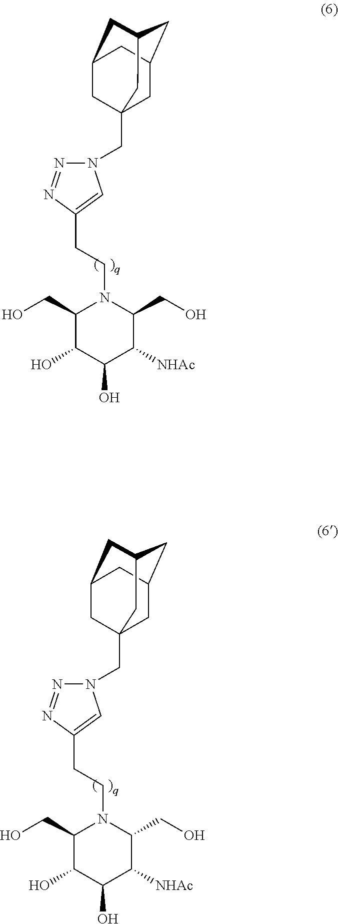

wherein m is an integer from 1 to 10 and q is an integer from 0 to 9. Preferably, m is 1, 2, 3 or 4. Ac represents —COCH3.

The compounds according to the present invention may be prepared by various methods known to those skilled in the art. More preferably, several chemical routes have been carried out. The present invention also concerns processes for preparing the compounds of the invention.

According to one embodiment, the present invention relates to a process for preparing a compound as defined above, more specifically compound (1′) or compounds of formula (I) with the same stereochemistry as compound (1′), wherein it comprises the steps of:
(i) hydrolysis of a compound of the following formula (III):

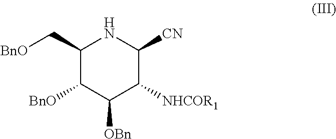

wherein R1 is as defined above, Bn represents a benzyl group or any other alcohol protecting group, followed by an esterification reaction, as to replace the nitrile function with an ester group,
(ii) reduction of the ester group as to obtain an alcohol group, and
(iii) optionally removing the alcohol protecting groups, preferably by hydrogenolysis.

The compound of formula (III) can be obtained for instance from N-acetyl-3,4,6-tri-O-benzylglucosamine by following the preparation as disclosed in A. Vasella, Helvetica Chimica Acta, 1998, 865.

The alcohol protecting groups are as defined above. Benzyl group is more particularly used in step (i) of the above described method.

Hydrolysis of step (i) is preferably carried out under basic conditions (pH above 7), more specifically at a pH around 14, obtained for instance with NaOH, KOH, LiOH, or CsOH. This reaction proceeds advantageously at a temperature ranging from 40 to 80° C., preferably at about 70° C.

Said hydrolysis is directly followed by an esterification reaction. The esterification reaction is implemented in presence of an alcohol, such as methanol, ethanol, or any other alcohol. The obtained ester may thus be a methyl, ethyl or any other ester. This reaction generally proceeds at room temperature (i.e.: 18° C.-25° C.).

The obtained ester can be extracted, washed, dried, and then purified, for instance by chromatography (e.g., chromatography on silica gel).

The formula of the obtained ester can be for instance formula (IIIa) below:

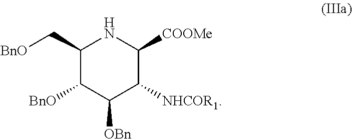

Step (ii) is implemented by any known means to reduce the ester group of the obtained compound as to obtain an alcohol group. This reaction generally proceeds at room temperature (i.e.: 18° C.-25° C.). More specifically, the ester is reduced with lithium or sodium borohydride. The reaction is carried in any suitable solvent such as ethanol. Preferably, the pH is maintained from 6 to 8.

The obtained alcohol can be extracted, washed, dried and then purified, for instance by chromatography (e.g., chromatography on silica gel).

When step (ii) is performed from compound of formula (IIIa), the obtained alcohol is of formula (IIIb) below:

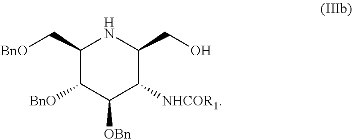

Step (ii) is preferably followed by step (iii) as to remove the alcohol protecting groups preferably by hydrogenolysis. Hydrogenolysis is preferably carried out by using palladium on activated carbon (Pd/C) with hydrogen. This reaction generally proceeds at room temperature (i.e., 18° C.-25° C.). The reaction is carried in any suitable solvent such as methanol.

The obtained product is preferably filtered and then evaporated to give rise to a solid.

Another object of the present invention is a compound of the formula (IIIa) or (IIIb).

According to another embodiment, the present invention relates to a process for preparing a compound as defined above, more specifically compound (1) or compounds of formula (I) with the same stereochemistry as compound (1), wherein it comprises the steps of:
(i) reacting a compound of the following formula (IV):

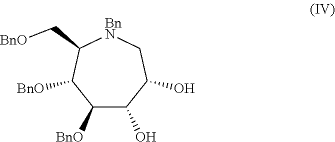

wherein Bn represents a benzyl group or any other suitable alcohol or amine protecting group respectively, so as to obtain a compound of the following formula (V):

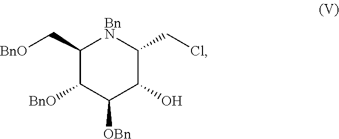 [SMILES: O[C@@H]1[C@@H](OCc2ccccc2)[C@H](OCc2ccccc2)[C@@H](COCc2ccccc2)N(Cc2ccccc2)[C@@H]1CCl]

(ii) preparing from compound of formula (V) a compound of formula (VI):

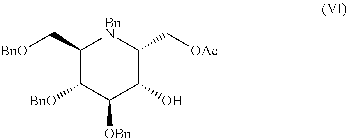 [SMILES: CC(=O)OC[C@@H]1[C@H](O)[C@@H](OCc2ccccc2)[C@H](OCc2ccccc2)[C@@H](COCc2ccccc2)N1Cc1ccccc1]

(iii) preparing from compound of formula (VI) a compound of formula (VII):

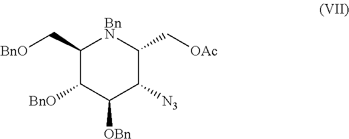 [SMILES: CC(=O)OC[C@@H]1[C@H](N=[N+]=[N-])[C@@H](OCc2ccccc2)[C@H](OCc2ccccc2)[C@@H](COCc2ccccc2)N1Cc1ccccc1]

(iv) preparing from compound of formula (VII) a compound of formula (VIII):

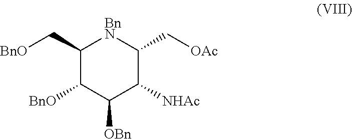 [SMILES: CC(=O)N[C@@H]1[C@@H](OCc2ccccc2)[C@H](OCc2ccccc2)[C@@H](COCc2ccccc2)N(Cc2ccccc2)[C@@H]1COC(C)=O]

(v) preparing from compound of formula (VIII) a compound of formula (IX):

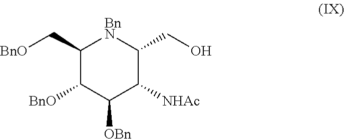 [SMILES: CC(=O)N[C@@H]1[C@@H](OCc2ccccc2)[C@H](OCc2ccccc2)[C@@H](COCc2ccccc2)N(Cc2ccccc2)[C@@H]1CO]

optionally removing the alcohol and amine protecting groups to obtain compound of formula (1):

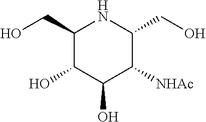 [SMILES: CC(=O)N[C@@H]1[C@@H](O)[C@H](O)[C@@H](CO)N[C@@H]1CO]

More specifically, step (i) is carried out by contacting compound of formula (IV) in a chlorinated solvent (such as dichloromethane) with tosylate chloride or preferably with mesylate chloride in a presence of a base, preferably Et₃N, pyridine, or diethylamine, more preferably Et₃N. Preferably, the reaction temperature is between −10° C. and +10° C., more preferably at about 0° C., and advantageously under inert atmosphere, such as argon. The compound of formula (IV) and preparation thereof are described in T. Liu, Y M Zhang, Y. Blériot, Synlett 2007, 905-908.

Alcohol or amine protecting groups are as defined above.

More specifically, step (ii) is carried out by contacting compound of formula (V) with silver acetate. Preferably, compound of formula (V) is in a polar aprotic solvent, such as dimethylformamide (or also called DMF).

Generally, step (iii) corresponds to a Mitsunobu reaction where, more specifically, PPh₃ and diisopropylazodicarboxylate (also called DIAD), or preferably diethylazodicarboxylate (also called DEAD), are added to a solution of compound of formula (VI) and then diphenylphosphoryl azide is added. Preferably, compound of formula (VI) is in a water-miscible organic solvent, such as tetrahydrofuran (also called THF). Preferably, the reaction is carried out at room temperature.

More specifically, according to step (iv), the azidopiperidine of formula (VII) obtained by step (iii) is reduced, in particular in presence of PPh₃, preferably in THF/H₂O. The reaction mixture is mores particularly then stirred at room temperature for several hours, in particular for 20-50 h (more specifically 40 hours), and at a temperature above 50° C. (more specifically at about 65° C.) for more than one hour, more specifically for 4 h, and then concentrated. The residue is preferably dissolved in EtOAc/H₂O. KHCO₃ and Ac₂O are then preferably added to the reaction mixture as to obtain compound of formula (VIII).

More specifically, according to step (v), hydrolysis of compound of formula (VIII) is performed to obtain compound of formula (IX). More specifically, KOH is added to a solution of compound of formula (VIII) in any suitable solvent, such as methanol.

Step (v) is preferably followed by step (vi) as to remove the alcohol and/or amine protecting groups preferably by hydrogenolysis. Hydrogenolysis is preferably carried out by contacting compound of formula (IX) with HCl. In another embodiment, hydrogenolysis is preferably carried out with palladium on activated carbon (Pd/C) with hydrogen. This reaction generally proceeds at room temperature (i.e., 18° C.-25° C.). The reaction is carried in any suitable solvent such as methanol.

The obtained product is preferably filtered and then evaporated to give rise to a solid.

It should be understood that other ways of producing these compounds may be designed by the skilled person, based on common general knowledge and following guidance contained in this application.

The products obtained by the above described methods may be used directly for biological applications or may be used for further synthesis to obtain other compounds, including compounds of formula (I) and (II), such as compounds with a substitution (R2 groups other than hydrogen atom) on the nitrogen atom of the piperidine ring and/or compounds with —CH2-R group replacing the —CH2OH group in position 6 of the piperidine ring. The compounds with —CH2-R group replacing the —CH2OH group in position 6 of the piperidine ring can be obtained through substitution of the chlorine atom in compound (V) in the presence of a base with various nucleophiles. The compounds with a substitution on the nitrogen atom of the piperidine ring can be obtained through substitution of the hydrogen atom on the nitrogen in compound of formula (I) with various nucleophiles. They can be prepared for instance by click connection with functionalised adamantanes or other groups as described by N. Ardes-Guisot et al., Org. Biomol. Chem., 2011, 9, 5373.

According to a particular embodiment, the compound of the invention is for use as a medicament. The present invention also provides a pharmaceutical composition comprising at least one compound as defined above in a pharmaceutically acceptable support.

The compound of the invention is more particularly for use in the treatment of type 2 diabetes, neurodegenerative diseases (such as Alzheimer disease), cancers, or viral diseases. According to another embodiment, the compound of the invention is more particularly for use in the treatment of lysosomal storage disorder, in particular Sanfilippo syndrome, Fabry disease, Tay-Sachs disease or Sandoff disease.

The compound of the invention can also be for use in the treatment of dyslipidaemia, haemostasis or fertility control.

According to another particular embodiment, the compound of the invention is for use as a biochemical tool, in particular to assess glycosidase mediated enzymatic hydrolysis reaction pathways and mechanisms.

Accordingly, it is herein disclosed a method for treating type 2 diabetes, neurodegenerative diseases (such as Alzheimer disease), cancers, viral diseases, lysosomal storage disorder, in particular Sanfilippo syndrome, Fabry disease, Tay-Sachs disease or Sandoff disease, which method comprises administering to a subject in need of such treatment an effective amount of at least one of compound of the invention.

It is also disclosed a method for treating dyslipidaemia, haemostasis or fertility control, which method comprises administering to a subject in need of such treatment an effective amount of at least one of compound of the invention.

The subject may be a human being or any animal, preferably a human being or a mammal, including cattle, sheep, horses, dogs, cats, goats etc. Poultry, fish or any other animals for food industry are also encompassed. Preferably the subject is a human patient, whatever his/her age or sex. New-borns, infants, children are included as well.

In the context of a treatment, the compounds of the invention may be administered to a subject by any suitable route, including oral, topical, sublingual, parenteral (preferably intravenous), transdermal, rectal, etc.

For a brief review of present methods for drug delivery, see, Langer, Science 249:1527-1533 (1990), which is incorporated herein by reference.

The present invention also concerns a pharmaceutical composition comprising a compound of the invention, in particular a compound of formula (I) or (II), as described above, and a pharmaceutically acceptable carrier and/or excipient. This particular aspect also concerns the preferred embodiments disclosed above for the compounds of the invention. In a particular embodiment, the pharmaceutical composition comprises a compound according to any of the above embodiments.

The pharmaceutical composition of the invention is formulated in accordance with standard pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art. The excipient of the composition can be any pharmaceutically acceptable excipient, including specific carriers able to target specific cells, cellular compartments or tissues. As stated earlier, possible pharmaceutical compositions include those suitable for oral, rectal, topical, transdermal, buccal, sublingual, or parenteral (including subcutaneous, intramuscular, intravenous and intradermal) administration. For these formulations, conventional excipients can be used according to techniques well known by those skilled in the art. The compositions for parenteral administration are generally physiologically compatible sterile solutions or suspensions which can optionally be prepared immediately before use from solid or lyophilized form. For oral administration, the composition can be formulated into conventional oral dosage forms such as tablets, capsules, powders, granules and liquid preparations such as syrups, elixirs, and concentrated drops. Non toxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatine, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants. For transdermal administration, the composition can be formulated into ointment, cream or gel form and appropriate penetrants or detergents could be used to facilitate permeation, such as dimethyl sulfoxide, dimethyl acetamide and dimethylformamide. For transmucosal administration, nasal sprays, rectal or vaginal suppositories can be used. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate. In a preferred embodiment, the pharmaceutical composition of the invention is suitable for parenteral administration.

Pharmaceutical composition according to the invention may be formulated to release the active drug substantially immediately upon administration or at any predetermined time or a time period after administration.

In a particular embodiment, the pharmaceutical composition according to the invention comprises 0.001 mg to 1 g of the compound of the invention. Preferably, pharmaceutical composition according to the invention comprises 0.01 mg to 800 mg of the compound of the invention.

Pharmaceutical compositions according to the invention can comprise one or more compound of the invention in association with pharmaceutically acceptable excipients and/or carriers. These excipients and/or carriers are chosen according to the form of administration as described above.

The below examples illustrate the invention without reducing its scope.

### EXAMPLES

### Example 1

### General

Optical rotations were measured at 20°±2° C. with a digital polarimeter by using a 10 cm, 1 mL cell. High-resolution mass spectrometry (HRMS) was carried out with a spectrometer in the positive ESI mode. NMR spectra were recorded with a spectrometer at ambient temperature (400 MHz). 1H NMR chemical shifts are referenced to residual protic solvent (CDCl₃; δH=7.28 ppm). 13C NMR chemical shifts are referenced to the solvent signal (δC=77.00 ppm for the central line of CDCl₃). Assignments were aided by the COSY, J-mod technique and HMQC. Reactions were monitored by thin-layer chromatography (TLC) on precoated silica gel 60 F254 plates (layer thickness 0.2 mm) and detected by charring with a 10% solution of CAN. Flash column chromatography was performed on silica gel 60 (230-400 mesh). Solvents were freshly distilled from Na/benzophenone (THF, toluene), or P₂O₅ (CH₂Cl₂).

### Synthesis of 3-Acetamido-4,5,7-tri-O-benzyl-2,3,6-trideoxy-2,6-imino-D-gulo-heptononitrile (III)

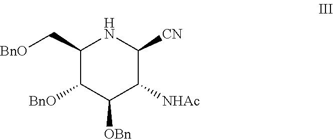 [SMILES: CC(=O)N[C@@H]1[C@@H](OCc2ccccc2)[C@H](OCc2ccccc2)[C@@H](COCc2ccccc2)N[C@H]1C#N]

The compound III was achieved using the procedure reported in the literature (A. Vasella, Helvetica Chimica ACTA, 1998, 865).

### Synthesis of (2R,3S,4R,5R,6R)-methyl 3-acetamido-4,5-dihydroxy-6-(hydroxymethyl)piperidine-2-carboxylate (IIIa)

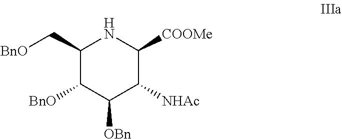 [SMILES: COC(=O)[C@@H]1N[C@H](COCc2ccccc2)[C@@H](OCc2ccccc2)[C@H](OCc2ccccc2)[C@H]1NC(C)=O]

To a solution of III (120 mg, 0.240 mmol) in MeOH/CHCl₃ 1:1 (25 mL) at room temperature, was added NaOH (192 mg, 4.81 mmol). The solution was warmed at 70° C. and left to react for 3 h. After that, the NaOH was quenched with HCl aq. solution and the product was extracted with AcOEt (3×50 mL), washed with brine (1×15 mL), dried on MgSO₄ and evaporated under vacuo. The crude product was purified by chromatography on silica gel (AcOEt/MeOH 9:1) gave 74 mg of yellow solid IIIa (59% yield). M.p. 149-150° C.; R_{f} 0.31 (AcOEt/MeOH 8:2). [α]_{D}²⁰=+20 (c 1.00, CHCl₃); ¹H-NMR (CDCl₃): 7.38-7.26 (m, 12H Bn), 7.22-7.10 (m, 3H Bn), 5.17 (d, J_{NH-C2}: 8.44, 1H), 4.86 (d, J: 11.76, 1H of CH₂-Bn on C3), 4.81 (d, J: 10.88, 1H of CH₂-Bn on C4), 4.64 (d, 1H of CH₂-Bn on C3), 4.52 (d, J: 11.8, 1H of CH₂-Bn on CH₂), 4.50 (d, 1H of CH₂-Bn on C4), 4.43 (d, 1H of CH₂-Bn on CH₂), 3.82 (m, 1H C2), 3.69 (m, 1H of CH₂), 3.68 (s, 3H OMe), 3.61 (m, 1H of CH₂), 3.59 (m, 1H C3), 3.56 (t, J: 8.2, 1H C4), 3.37 (d, J_{C1-C2}: 10.3, 1H Cl), 2.74 (m, 1H C5), 1.76 (s, 3H CH₃); ¹³C-NMR (CDCl₃): 171.19 (C_{q} COOMe), 170.36 (C_{q} NHCOMe), 138.53 (C_{q} Bn), 138.27 (C_{q} Bn), 138.01 (C_{q} Bn), 128.80 (2×CH Bn), 128.65 (2×CH Bn), 128.64 (2×CH Bn), 128.53 (2×CH Bn), 128.23 (2×CH Bn), 128.17 (1×CH Bn), 128.15 (2×CH Bn), 128.02 (2×CH Bn), 82.82 (C3), 80.57 (C4), 75.28 (CH₂-Bn on C3) 74.80 (CH₂-Bn on C4), 73.62 (CH₂-Bn on the CH₂), 69.16 (CH2), 61.53 (C1), 58.56 (C5), 55.25 (C2), 52.70 (OMe), 23.46 (CH₃); ESI-Mass: 533.0720 (M+H, C₃₁H₃₇N₂O₆ requires 533.2646), 555.2474, (M+Na). IR: 3284, (stretching NH), 2923-2853, 1736 (stretching COOMe), 1651 (stretching NHCOCH₃), 1551, 1454, 1095, 748, 697.

### Synthesis of N-((2R,3R,4R,5S,6R)-1-benzyl-3,4-bis(benzyloxy)-2-((benzyloxy)methyl)-6-(hydroxymethyl)piperidin-5-yl)acetamide (IIIb)

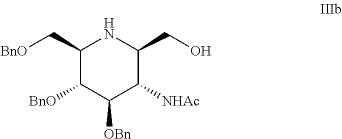 [SMILES: CC(=O)N[C@@H]1[C@@H](OCc2ccccc2)[C@H](OCc2ccccc2)[C@@H](COCc2ccccc2)N[C@H]1CO]

To a solution of IIIa (30 mg, 0.057 mmol) at 0° C. the NaBH₄ (21.4 mg, 0.565 mmol) was added. The pH of suspension was adjusted 7.5-8.0 by dropwising HCl 1M solution. The white suspension was left to react at 0° C. for 1 h and then the NaBH₄ (10.7 mg, 0.285 mmol) was added and the pH was again adjusted with HCl solution. After another 1 h, the NaBH₄ (10.7 mg, 0.285 mmol) was added, the pH was adjusted and the reaction was left to react at 0° C. for another 1 h. At this point, the NH₄Cl aq. (20 mL) solution was added and the product was extracted with AcOEt (3×30 mL), washed with brine (1×10 mL), dried on MgSO₄ and evaporated under vacuo. The crude product was purified by chromatography on silica gel (AcOEt/MeOH 8:2) gave 25.3 mg of white solid IIIb (88% yield). M.p. 206° C. R_{f} 0.28 (AcOEt/MeOH 7:3). [α]_{D}²⁰=+30 (c 1.00, CHCl₃); ¹H-NMR (CDCl₃): 7.42-7.27 (m, 12H Bn), 7.25-7.21 (m, 3H Bn), 5.29 (s, 1H NH), 4.89 (d, J: 12.1, 1H of CH₂-Bn on C3), 4.83 (d, J: 10.8, 1H of CH₂-Bn on C4), 4.65 (d, 1H of CH₂-Bn on C3), 4.53 (d, 2H overlap of CH₂-Bn on C4 and CH₂), 4.45 (d, J: 11.9, 1H of CH₂-Bn on CH₂), 3.72 (m, 1H C2), 3.69 (d.d, J₁: 2.44, J₂: 9.1, 1CH of CH₂), 3.56 (t, J_{C3-C4}=J_{C3-C5}: 9.0, 1H C4), 3.52 (d, J_{CH2OH-C1}: 1.96, 2H of CH₂OH), 3.41 (d.d, J_{C3-C2}: 8.8, 1H C3), 2.74 (m, 1H C5), 2.26 (d, J_{C1-C2}: 10-3, 1H C1), 1.75 (s, 3H CH₃); ¹³C-NMR (CDCl₃): 172.22 (C_{q} NHCOMe), 138.62 (C_{q} Bn), 138.26 (C_{q} Bn), 138.08 (C_{q} Bn), 129.09 (2×CH Bn), 128.94 (2×CH Bn), 128.70 (2×CH Bn), 128.66 (2×CH Bn), 128.50 (1×CH Bn), 128.34 (2×CH Bn), 128.24 (2×CH Bn), 128.10 (1×CH Bn), 128.01 (1×CH Bn), 82.78 (C3), 81.20 (C4), 75.30 (CH₂-Bn on C4) 74.22 (CH₂-Bn on C3), 73.70 (CH₂-Bn on the CH₂), 69.28 (CH₂—OBn), 62.21 (CH₂OH), 60.97 (C1), 59.18 (C5), 52.57 (C2), 23.24 (CH₃); ESI-Mass: 505.2711 (M+H, C₃₀H₃₇N₂O₅ requires 505.2697), 527.2523, (M+Na); IR: 3280 (stetching OH), 2923-2853, 1650 (stretching CO), 1560, 1453, 1099, 742, 695.

### Synthesis of N-((2R,3R,4R,5S,6R)-3,4-dihydroxy-2,6-bis(hydroxymethyl)piperidin-5-yl)acetamide (1β)

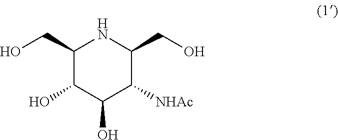 [SMILES: CC(=O)N[C@@H]1[C@@H](O)[C@H](O)[C@@H](CO)N[C@H]1CO]

To a solution of IIIb (30 mg, 0.059 mmol) in MeOH (3 mL) at r.t., the Pd/C was added. The black suspension was treated by hydrogen for 24 h. After that, the product was filtrated on 0.45 μm filter and evaporated under vacuo to give 12.3 mg of white solid 1′ (89% yield). R_{f} 0.65 (AcOEt/MeOH 1:1). [α]_{D}²⁰=+2 (c 1.00, MeOH); ¹H-NMR (CDCl₃): 3.89 (d.d, J_{C6-CH2}: 12.0, J: 3.1 of diasterotopic proton of CH₂ on C6, 1H), 3.61 (d.d, J: 2.6, J: 8.4, 1H C), 3.59 (m, 1H of CH₂ on C1), 3.56 (t, J_{C3-C2}=J_{C3-C4}: 10.5, 1H C3), 3.46 ((d.d, J_{C6-CH2}: 11.5, 1H of CH₂ on C6) 3.33 (d.d, J_{C4-C3}: 8.8, 1H C4), 3.18 (d.d, J_{C5-C6}: 8.8, 1H C5), 2.52-2.58 (m, 2H C1 and C6); ¹³C-NMR (CDCl₃): 174.24 (Cq NHCOMe), 77.88 (C4), 74.17 (C5), 63.50 (CH₂ on C1), 63.30 (CH₂ on C6), 61.88 (CH₂ on C6), 61.33 (CH₂ on C1), 54.53 (C3), 22.69 (CH₃); ESI-Mass: 257.1118 (M+Na, C₉H₁₈N₂NaO₅ requires 257.1108), 491.2336 (2M+Na); IR: 3283 (stretching OH), 2863, 1639, 1533, 1445, 1114, 739, 697.

### Example 2

### Synthesis of (2R,3R,4R,5S,6S)-1-benzyl-3,4-bis(benzyloxy)-2-((benzyloxy)methyl)azepane-5,6-diol (IV)

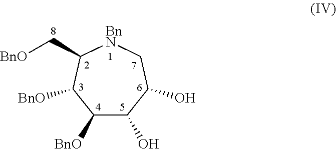 [SMILES: O[C@@H]1[C@@H](OCc2ccccc2)[C@H](OCc2ccccc2)[C@@H](COCc2ccccc2)N(Cc2ccccc2)C[C@@H]1O]

The compound of formula (IV) was prepared as described in T. Liu, Y M Zhang, Y. Blériot, Synlett 2007, 905-908.

¹H-NMR (CDCl₃): 7.45-7.18 (m, 20H, Hₐᵣ), 4.84, 4.65 (2d, 2H, ²J=11.3 Hz, CH₂Ph), 4.75, 4.42 (2d, 2H, ²J=11.2 Hz, CH₂Ph), 4.49, 4.44 (2d, 2H, ²J=12.0 Hz, CH₂Ph), 3.99 (d, 1H, ²J=13.4 Hz, NCHPh), 3.92 (dd, 1H, J=7.5 Hz, J=7.4 Hz, H₄), 3.89 (ddd, 1H, J_{H6-H5}=5.8 Hz, J_{H6-H7b}=5.8 Hz, J_{H6-H7a}=2.7 Hz, H₆), 3.79 (d, 1H, ²J=13.4 Hz, NCHPh), 3.76-3.66 (m, 4H, H₃, H₅, H₈), 3.35 (dd, 1H, J_{H7a-H7b}=14.4 Hz, J_{H7a-H6}=2.7 Hz, H₇ₐ), 3.10 (ddd, 1H, J=7.2 Hz, J=4.1 Hz, J=3.7 Hz, H₂), 2.80 (dd, 1H, J_{H7b-H7a}=14.4 Hz, J_{H7b-H6}=5.8 Hz, H_{7b}); ¹³C-NMR (CDCl₃): 139.3, 138.3, 138.3, 138.2 (Cᵢₚₛₒ), 129.0, 128.7, 128.6, 128.5, 128.5, 128.1, 128.0, 127.9, 127.8, 127.8, 127.5 (CHₐᵣ), 82.4 (C₄), 79.6 (C₃), 76.6 (C₅), 74.6, 74.0, 73.4 (CH₂Ph), 69.1 (C₆), 67.3 (C₈), 62.5 (C₂), 60.8 (NCH₂Ph), 52.0 (C₇).

### Synthesis of (2R,3R,4R,5S,6S)-1-benzyl-3,4-bis(benzyloxy)-2-((benzyloxy)methyl)-6-(chloromethyl)-5-hydroxypiperidine (V)

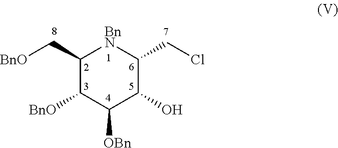 [SMILES: O[C@@H]1[C@@H](OCc2ccccc2)[C@H](OCc2ccccc2)[C@@H](COCc2ccccc2)N(Cc2ccccc2)[C@@H]1CCl]

To a stirred solution of diol (IV)* (856 mg, 1.55 mmol) in dry DCM (20 mL) was added Et₃N (650 μL, 4.65 mmol, 3 eq.) at 0° C. under argon, followed by addition of MsCl (155 μL, 2.01 mmol, 1.3 eq.). The reaction mixture was stirred at 0° C. for 1 h, directly transferred to a dry silica gel column and eluted with PE/EtOAc (100:0 to 90:10) to afford chloromethyl piperidine (V) (293 mg, 33%, clear oil). R_{f} 0.35 (PE/EtOAc=9:1); ¹H-NMR (CDCl₃): 7.42-7.22 (m, 20H, Hₐᵣ), 4.67, 4.61 (2d, 2H, ²J=11.7 Hz, CH₂Ph), 4.54, 4.51 (2d, 2H, ²J=11.5 Hz, CH₂Ph), 4.40 (s, 2H, CH₂Ph), 4.07 (s, 2H, NCH₂Ph), 3.95-3.89 (m, 1H, H₅), 3.87 (dd, 1H, J_{H8a-H8b}=9.8 Hz, J_{H8a-H2}=5.3 Hz, H₈ₐ), 3.85-3.70 (m, 5H, H₃, H₄, H₅, H₇), 3.38 (ddd, 1H, J=6.5 Hz, J=6.2 Hz, J=2.9 Hz, H₆), 3.31 (ddd, 1H, J_{H2-H8a}=5.3 Hz, J_{H2-H8b}=5.2 Hz, J_{H2-H3}=5.2 Hz, H₂), 3.15 (d, 1H, J_{OH-H5}=8.5 Hz, OH); ¹³C-NMR (CDCl₃): 140.6, 138.3, 138.1, 137.9 (Cᵢₚₛₒ), 128.6, 128.6, 128.5, 128.4, 128.3, 128.0, 127.9, 127.8, 127.7, 127.0 (CHₐᵣ), 78.7 (C₄), 76.0 (C₃), 73.2, 73.2, 72.6 (CH₂Ph), 69.2 (C₅), 66.9 (C₈), 58.7 (C₆), 58.6 (C₂), 53.8 (NCH₂Ph), 43.0 (C₇); HRMS calcd for C₃₅H₃₉ClNO₄⁺ 572.2562, found 572.2588.

*ref: T. Liu, Y M Zhang, Y. Blériot, Synlett 2007, 905-908

### Synthesis of (2R,3R,4R,5S,6R)-1-benzyl-3,4-bis(benzyloxy)-2-((benzyloxy)methyl)-5-hydroxypiperidin-6-yl)methyl acetate (VI)

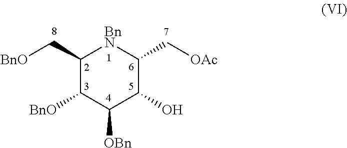 [SMILES: CC(=O)OC[C@@H]1[C@H](O)[C@@H](OCc2ccccc2)[C@H](OCc2ccccc2)[C@@H](COCc2ccccc2)N1Cc1ccccc1]

To a stirred solution of chloropiperidine (V) (289 mg, 0.51 mmol) in dry DMF (6 mL) was added silver (I) acetate (127 mg, 0.76 mmol, 1.5 eq.). The reaction mixture was stirred at 60° C. overnight, diluted with EtOAc and washed with saturated aqueous NaHCO₃ solution, dried (MgSO₄) and concentrated. Purification by flash chromatography (PE/EtOAc, 8:2) afforded acetate (VI) (168 mg, 55%, pale yellow oil). [α]_{D}²⁰=+2 (c 1.68, CHCl₃); ¹H-NMR (CDCl₃): 7.39-7.19 (m, 20H, Hₐᵣ), 4.71, 4.61 (2d, 2H, ²J=11.7 Hz, CH₂Ph), 4.55, 4.47 (2d, 2H, ²J=11.3 Hz, CH₂Ph), 4.39 (dd, 1H, J_{H7a-H7b}=11.8 Hz, J_{H7a-H6}=7.0 Hz, H₇ₐ), 4.38 (s, 2H, CH₂Ph), 4.29 (dd, 1H, J_{H7b-H7a}=11.8 Hz, J_{H7b-H6}=4.4 Hz, H_{7b}), 4.00, 3.97 (2d, 2H, ²J=14.2 Hz, NCH₂Ph), 3.87 (dd, 1H, J_{H8a-H8b}=10.0 Hz, J_{H8a-H2}=5.2 Hz, H₈ₐ), 3.84-3.73 (m, 3H, H₅, H₃, H_{8b}), 3.68 (dd, 1H, J=6.7 Hz, J=5.8 Hz, H₄), 3.29 (ddd, 1H, J_{H6-H7a}=7.0 Hz, J_{H6-H7b}=4.4 Hz, J_{H6-H5}=4.0 Hz, H₆), 3.23 (ddd, 1H, J_{H2-H8a}=5.2 Hz, J=5.2 Hz, J=5.0 Hz, H₂), 2.05 (s, 3H, Ac); ¹³C-NMR (CDCl₃): 170.9 (CO), 140.4, 138.3, 138.1 (Cᵢₚₛₒ), 128.7, 128.5, 128.5, 128.5, 128.3, 128.0, 127.8, 127.8, 127.7, 127.7, 127.0 (CHₐᵣ), 80.0 (C₄), 76.5 (C₃), 73.6, 73.2, 72.9 (CH₂Ph), 69.8 (C₅), 66.7 (C₈), 62.8 (C₇), 58.1 (C₂), 56.0 (C₆), 53.2 (NCH₂Ph), 21.3 (Ac); HRMS calcd for C₃₇H₄₂NO₆⁺ 596.3007, found 596.3026.

### Synthesis of ((2R,3R,4R,5S,6S)-5-azido-1-benzyl-3,4-bis(benzyloxy)-2-((benzyloxy)methyl)piperidin-6-yl)methyl acetate (VII)

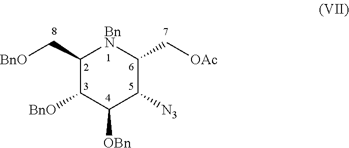 [SMILES: CC(=O)OC[C@@H]1[C@H](N=[N+]=[N-])[C@@H](OCc2ccccc2)[C@H](OCc2ccccc2)[C@@H](COCc2ccccc2)N1Cc1ccccc1]

To a stirred solution of (VI) (65 mg, 0.11 mmol) in dry THF (1 mL) were added PPh₃ (57 mg, 0.22 mmol, 2 eq.) and DEAD (100 μL, 0.22 mmol, 2 eq.). After 5 min stirring at r.t., diphenylphosphoryl azide (240 μL, 1.1 mmol, 10 eq.) was added and the reaction mixture was stirred overnight at r.t., then concentrated. Purification by flash chromatography (PE/EtOAc, 9:1) afforded azidopiperidine (VII) (51 mg, 75%, pale yellow oil). [α]_{D}²⁰=+27 (c 1.06, CHCl₃); ¹H-NMR (CDCl₃): 7.40-7.20 (m, 20H, Hₐᵣ), 4.86, 4.61 (2d, 2H, ²J=10.9 Hz, CH₂Ph), 4.85 (s, 2H, CH₂Ph), 4.39 (dd, 1H, J_{H7a-H7b}=11.9 Hz, J_{H7a-H6}=7.5 Hz, H₇ₐ), 4.38, 4.34 (2d, 2H, ²J=11.8 Hz, CH₂Ph), 4.17 (dd, 1H, J_{H7b-H7a}=11.9 Hz, J_{H7-H6}=3.6 Hz, H_{7b}), 4.04, 3.94 (2d, 2H, ²J=14.9 Hz, NCH₂Ph), 3.84 (dd, 1H, J_{H8a-H8b}=10.5 Hz, J_{H8a-H2}=4.2 Hz, H₈ₐ), 3.79 (dd, 1H, J_{H3-H2}=10.0 Hz, J_{H3-H4}=8.8 Hz, H₃), 3.78 (dd, 1H, J_{H5-H4}=10.1 Hz, J_{H5-H6}=6.0 Hz, H₅), 3.71 (dd, 1H, J_{H8b-H8a}=10.5 Hz, J_{H8b-H2}=1.8 Hz, H_{8b}), 3.62 (dd, 1H, J J_{H4-H5}=10.1 Hz, J_{H4-H3}=8.8 Hz, H₄), 3.17 (ddd, 1H, J_{H6-H7a}=7.5 Hz, J_{H6-H5}=6.0 Hz, J_{H6-H7b}=3.6 Hz, H₆), 3.10 (ddd, 1H, J_{H2-H3}=10.0 Hz, J_{H2-H8a}=4.2 Hz, J_{H2-H8b}=1.8 Hz, H₂), 2.05 (s, 3H, Ac); ¹³C-NMR (CDCl₃): 170.8 (CO), 140.0, 138.5, 138.0, 138.0 (Cᵢₚₛₒ), 128.6, 128.5, 128.4, 128.4, 128.2, 128.0, 127.8, 127.8, 127.7, 127.1 (CHₐᵣ), 83.8 (C₄), 79.0 (C₃), 75.6, 75.1, 73.1 (CH₂Ph), 67.8 (C₈), 62.4 (C₅), 59.3 (C₇), 58.7 (C₂), 57.4 (C₆), 52.8 (NCH₂Ph), 21.2 (Ac); HRMS calcd for C₃₇H₄₀N₄NaO₅⁺ 643.2891, found 643.2916.

### Synthesis of ((2R,3R,4R,5S,6S)-5-acetamido-1-benzyl-3,4-bis(benzyloxy)-2-((benzyloxy)methyl)piperidin-6-yl)methyl acetate (VIII)

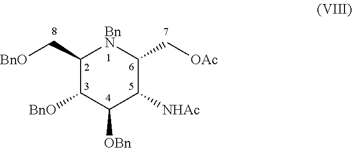 [SMILES: CC(=O)N[C@@H]1[C@@H](OCc2ccccc2)[C@H](OCc2ccccc2)[C@@H](COCc2ccccc2)N(Cc2ccccc2)[C@@H]1COC(C)=O]

To a stirred solution of azidopiperidine (VII) (183 mg, 0.29 mmol) in THF/H₂O (15 mL/1 mL) was added PPh₃ (193 mg, 0.74 mmol, 2.5 eq.). The reaction mixture was stirred at 65° C. for 4 h, and concentrated. The residue was dissolved in pyridine (2 mL). Ac₂O (2 mL) was added and the reaction mixture was stirred at r.t. for 3 h, diluted with EtOAc and washed with brine, dried (MgSO₄) and concentrated. Purification by flash chromatography (PE/EtOAc, 6:4) afforded acetamide (VIII) (176 mg, 94%, clear oil). [α]_{D}²⁰=−10 (c 0.62, CHCl₃); ¹H-NMR (CDCl₃): 7.38-7.21 (m, 20H, Hₐᵣ), 6.55 (d, 1H, J_{NH-H5}=9.0 Hz, NH), 4.66, 4.57 (2d, 2H, ²J=11.6 Hz, CH₂Ph), 4.43, 4.38 (2d, 2H, ²J=12.1 Hz, CH₂Ph), 4.43-4.30 (m, 1H, H₅), 4.40, 4.33 (2d, 2H, ²J=11.2 Hz, CH₂Ph), 4.16 (dd, 1H, J_{H7a-H7b}=11.8 Hz, J_{H7a-H6}=6.2 Hz, H₇ₐ), 4.06 (dd, 1H, J_{H7b-H7a}=11.8 Hz, J_{H7b-H6}=5.2 Hz, H_{7b}), 3.96, 3.86 (2d, 2H, ²J=14.2 Hz, NCH₂Ph), 3.90 (dd, 1H, J_{H8a-H8b}=9.7 Hz, J_{H8a-H2}=5.5 Hz, H₈ₐ), 3.80 (dd, 1H, J_{H8b-H8a}=9.7 Hz, J_{H8b-H2}=7.0 Hz, H_{8b}), 3.79-3.74 (m, 1H, H₃), 3.66 (dd, 1H, J=3.5 Hz, J=3.3 Hz, H₄), 3.51-3.50 (m, 1H, H₆), 3.37-3.31 (m, 1H, H₂), 2.03 (s, 3H, OAc), 1.78 (s, 3H, NHAc); ¹³C-NMR (CDCl₃): 170.7 (CH₃CONH), 169.7 (CH₃COO), 140.4, 138.4, 138.1, 138.1 (Cᵢₚₛₒ), 128.6, 128.5, 128.5, 128.4, 128.3, 127.9, 127.7, 127.7, 127.6, 127.5, 127.1 (CHₐᵣ), 76.4 (C₄), 75.4 (C₃), 73.3, 72.3, 71.7 (CH₂Ph), 66.0 (C₈), 64.2 (C₇), 58.3 (C₂), 53.4 (NCH₂Ph), 52.9 (C₆), 48.3 (C₅), 23.5 (NHAc), 21.1 (OAc); HRMS calcd for C₃₉H₄₄N₂NaO₆⁺ 659.3092, found 659.3108.

### Synthesis of N-((2R,3R,4R,5S,6S)-1-benzyl-3,4-bis(benzyloxy)-2-((benzyloxy)methyl)-6-(hydroxymethyl)piperidin-5-yl)acetamide (IX)

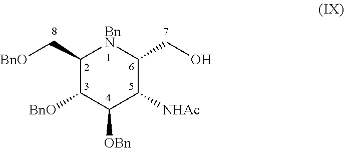 [SMILES: CC(=O)N[C@@H]1[C@@H](OCc2ccccc2)[C@H](OCc2ccccc2)[C@@H](COCc2ccccc2)N(Cc2ccccc2)[C@@H]1CO]

To a stirred solution of acetate (VIII) (17 mg, 27 μmol) in MeOH (1 mL) was added KOH (3 mg, 53 μmol, 2 eq.). The reaction mixture was stirred at r.t. for 1 h, diluted with brine and extracted with EtOAc. The organic layer was dried (MgSO₄) and concentrated. Purification by flash chromatography (EtOAc/PE, 2:1) afforded (hydroxymethyl)piperidine (IX) (14.6 mg, 91%, clear oil). [α]_{D}²⁰=−5 (c 0.36, CHCl₃); ¹H-NMR (CDCl₃): 7.41-7.18 (m, 21H, Hₐᵣ, NH), 4.64, 4.51 (2d, 2H, ²J=11.8 Hz, CH₂Ph), 4.47, 4.31 (2d, 2H, ²J=11.1 Hz, CH₂Ph), 4.43, 4.39 (2d, 2H, ²J=12.1 Hz, CH₂Ph), 4.39-4.31 (m, 1H, H₅), 3.89 (dd, 1H, J_{H8a-H8b}=9.5 Hz, J_{H8a-H2}=5.7 Hz, H₈ₐ), 3.86-3.68 (m, 6H, NCH₂Ph, H₃, H₄, H₇ₐ, H_{8b}), 3.45-3.38 (m, 1H, H₆), 3.38-3.32 (m, 1H, H₂), 3.20 (m, 1H, H_{7b}), 1.86 (s, 3H, Ac); ¹³C-NMR (CDCl₃): 171.2 (CO), 140.9, 138.4, 137.9, 137.8 (Cᵢₚₛₒ), 128.7, 128.6, 128.5, 128.3, 128.2, 128.1, 127.9, 127.7, 127.7, 127.6, 127.0 (CHₐᵣ), 75.8 (C₄), 75.2 (C₃), 73.4, 72.0, 71.6 (CH₂Ph), 65.9 (C₈), 60.7 (C₇), 58.4 (C₂), 54.7 (C₆), 53.3 (NCH₂Ph), 46.9 (C₅), 23.2 (Ac); HRMS calcd for C₃₇H₄₂N₂NaO₅⁺ 617.2986, found 617.3004.

### Synthesis of N-((2R,3R,4R,5S,6S)-3,4-dihydroxy-2,6-bis(hydroxymethyl)piperidin-5-yl)acetamide (1)

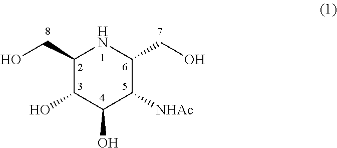 [SMILES: CC(=O)N[C@@H]1[C@@H](O)[C@H](O)[C@@H](CO)N[C@@H]1CO]

Piperidine (IX) (12.4 mg, 21 μmol) was dissolved in MeOH (2 mL) and 1M aq. HCl (25 μL). 10% Pd/C (24 mg) was added. The suspension was stirred under H₂ for 48 hours at r.t., filtered through Celite and eluted with MeOH. The solvent was removed under reduced pressure to afford piperidine (1) (quant. yield) as its hydrochloride salt. [α]_{D}²⁰=+47 (c 0.17, MeOH); ¹H-NMR (CD₃OD): 4.01 (dd, 1H, J_{H5-H4}=10.5 Hz, J_{H5-H6}=5.6 Hz, H₅), 3.89 (dd, 1H, J_{H8a-H8b}=10.7 Hz, J_{H8a-H2}=3.2 Hz, H₈ₐ), 3.74 (dd, 1H, J_{H7a-H7b}=11.0 Hz, J_{H7a-H6}=10.5 Hz, H₇ₐ), 3.63-3.55 (m, 2H, H_{7b}, H_{8b}), 3.52 (dd, 1H, J_{H4-H5}=10.5 Hz, J_{H4-H3}=8.3 Hz, H₄), 3.37 (ddd, 1H, J_{H6-H7a}=10.5 Hz, J_{H6-H5}=5.6 Hz, J_{H6-H7b}=5.1 Hz, H₆), 3.23 (dd, 1H, J_{H3-H2}=9.3 Hz, J_{H3-H4}=8.3 Hz, H₃), 2.90 (m, 1H, H₂), 1.98 (s, 3H, Ac); ¹³C-NMR (CD₃OD): 74.2 (C₃), 73.1 (C₄), 63.1 (C₈), 58.5 (C₇), 56.5 (C₂), 56.2 (C₆), 54.2 (C₅), 22.7 (Ac); HRMS calcd for C₉H₁₉N₂O₅⁺ 235.1288, found 235.1302.

### Example 3

### In Vitro Glycosidase Inhibitory Activities

IC50 values of compounds of the invention towards various glycosidases are shown in Table 1 below.

All glycosidases and p-nitrophenyl glycosides were purchased from Sigma-Aldrich Co. The enzyme assays were performed according to the methods described in Kato, A.; Miyauchi, S.; Kato, N.; Nash, R. J.; Yoshimura, Y.; Nakagome, I.; Hirono, S.; Takahata, H.; Adachi, I. Bioorg. Med. Chem. 19, 3558-3568, 2011. The activities were determined using an appropriate p-nitrophenyl glycoside as substrate at the optimum pH of each enzyme. The reaction mixture contained 2 mM of the substrate and the appropriate amount of enzyme. The reaction was stopped by adding 2 mL of 400 mM Na₂CO₃. The released p-nitrophenol was measured spectrometrically at 400 nm.

TABLE 1

| | IC₅₀ (μM) | |
|---|---|---|
| | Compound β | Compound α |
| enzyme | (1′) | (1) |
| α-glucosidase | | |
| yeast | NI^{a} (15.4%)^{b} | NI (18.5%) |
| rice | NI (15.9%) | NI (32.3%) |
| rat intestinal maltase | NI (1.3%) | NI (26.7%) |
| β-glucosidase | | |
| almond | NI (18.3%) | NI (5.0%) |
| bovine liver | NI (15.5%) | NI (4.4%) |
| α-galactosidase | | |
| coffee beans | NI (13.1%) | NI (19.7%) |
| β-galactosidase | | |
| bovine liver | NI (5.7%) | NI (5.0%) |
| α-mannosidase | | |
| jack beans | NI (0%) | NI (2.3%) |
| β-mannosidase | | |
| snail | NI (0%) | NI (1.9%) |
| β-N-acetyl hexosaminidase | | |
| human placenta | 72 | 56 |
| bovine kidney | 65 | 67 |
| α-N-acetyl galactosaminidase | | |
| chicken liver | NI (42.7%) | NI (42.4%) |

^{a}NI: No Inhibition (less than 50% inhibition at 1000 μM).
^{b}% of inhibition at 1000 μM.

Compounds (1) and (1′) present a selective inhibitory activity towards β-N-acetyl hexosaminidase, which is of particular interest for drug development.

### Example 4

### Enzyme Enhancement in Sanfillipo Cells

Compounds (1) and (B) (compound (B) is described in A. Vasella, Helvetica Chimica Acta, 1998, 865) were tested in 3 Sanfillipo patient-derived fibroblast cell lines, named as MPS IIIB fibroblast cells lines below.

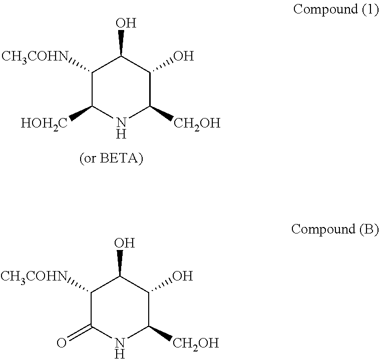

### Enzyme Activation Assay

MPS IIIB fibroblasts (GM01426, GM02931 and GM00737) were cultured in the presence of various concentrations of compounds (1) and (B) (0-10 μM) for 3 days before α-N-acetylglucosaminidase (NAGLU) activity was measured in cell homogenates using 4-methylumbelliferyl-α-N-acetylglucosamine (4-MU-α-GlcNAc) as substrate. Cells were washed twice in phosphate buffered saline, homogenised in water using a small dounce homogeniser, centrifuged at 800 g for 5 min and the supernatant taken for protein and enzyme activity. Protein concentration was determined using the BCA assay (Pierce, UK) according to manufacturer's instructions. An aliquot, (5 μl) of homogenate was added to a well of a 96-well plate containing 10 μl 2 mM 4-MU substrate (in water) and 5 μl 0.2 M sodium acetate buffer, pH 4.3. Following incubation for 3-4 h at 37° C. in a humidified incubator, the reaction was stopped by adding 300 μl 0.5 M glycine/NaOH, pH 10.3 and the fluorescence measured at 460 nm using an excitation wavelength of 350 nm, em 460 nm. Enzyme activation is defined as the fold increase in fluorescence due to enzyme activity (FU/μg protein) in treated cells compared to untreated cells. All assays were performed in triplicate, mean and error bars (standard deviations) are shown.

### Results: Compounds (1) and (B)

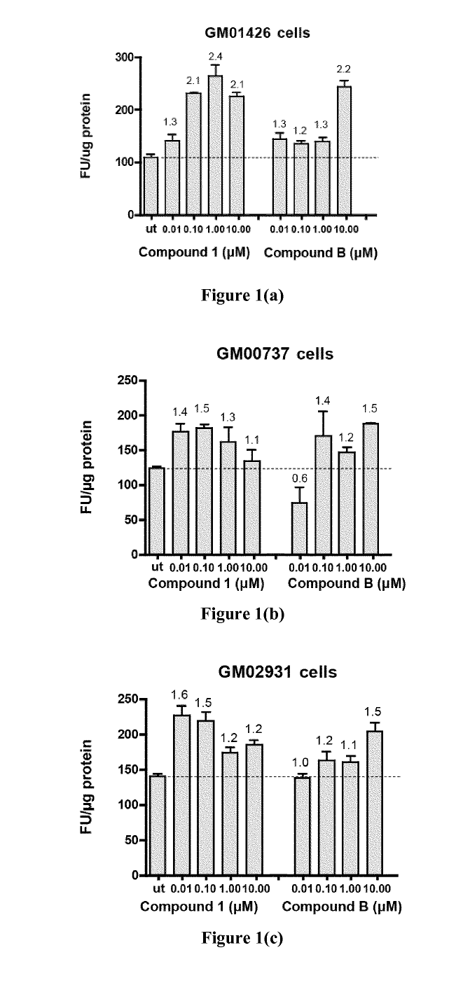

Results are given in FIG. 1(a)-(c).

Compound (1) has a differential activity in cell lines, either showing a concentration dependent increase up to 1 μM in GM01426 (the best activation seen to date was 2.4 fold) or activation at lower concentrations in GM00737 and GM02931 cells. The effects of compound (B) are weaker and possibly require higher concentrations.

In view of these results, Compound (1) appears particularly useful in treating a lysosomal storage disorder, in particular Sanfilippo syndrome.

Patents, patent applications, publications, product descriptions, and protocols which are cited throughout this application are incorporated herein by reference in their entireties for all purposes.

To facilitate an understanding of the principles and features of the invention, various illustrative embodiments are described in this specification. Although exemplary embodiments are explained in detail, it is to be understood that other embodiments are contemplated. Accordingly, neither the invention, nor any of the appended claims, is limited in its scope to specific examples or embodiments herein, or to the details of construction and arrangement of components set forth in the foregoing description or illustrated in the drawings. The invention is capable of other embodiments and of being practiced, carried out, and claimed in various ways.

1. A compound of general formula (I): 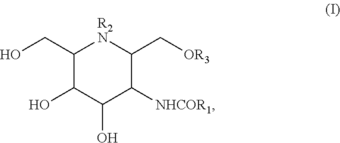
wherein:
R1 represents an alkyl (C1-C10) group, an alkenyl (C2-C10) group, an alkynyl (C2-C10) group, a cycloalkyl (C3-C10) group, a heterocycle (C3-C18) group, an aryl (C6-C18) group, or an arylalkyl group;
R2 represents a hydrogen atom, an alkyl (C1-C10) group, an alkenyl (C2-C10) group, an alkynyl (C2-C10) group, a cycloalkyl (C3-C10) group, a heterocycle (C3-C18) group, an aryl (C6-C18) group, an arylalkyl group, or a heteroaryl (C4-C16) group; and
R3 represents a hydrogen atom, an alkyl (C1-C10) group, an alkenyl (C2-C10) group, an alkynyl (C2-C10) group, a cycloalkyl (C3-C10) group, a heterocycle (C3-C18) group, an aryl (C6-C18) group, or an arylalkyl group; or
any geometrical or optical isomer thereof.

2. The compound according to claim 1, wherein the compound is of the following formula:

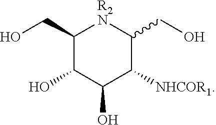

3. The compound according to claim 1, wherein the compound is of the following formula:

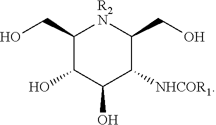

4. The compound according to claim 1, wherein the compound is of the following formula:

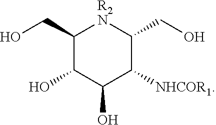

5. The compound according to claim 1, wherein R2 is a hydrogen atom, an alkyl (C1-C8) group, a cycloalkyl (C3-C10) group, or an alkyl (C8-C10) group substituted with at least one heterocycle, cycloalkyl (C3-C10) group or alkoxy group.

6. The compound according to claim 1, wherein R1 represents an alkyl (C1-C8) group, a cycloalkyl (C3-C10) group, or a heterocycle (C3-C18) group.

7. The compound according to claim 1, wherein the compound is one of the following formulae: 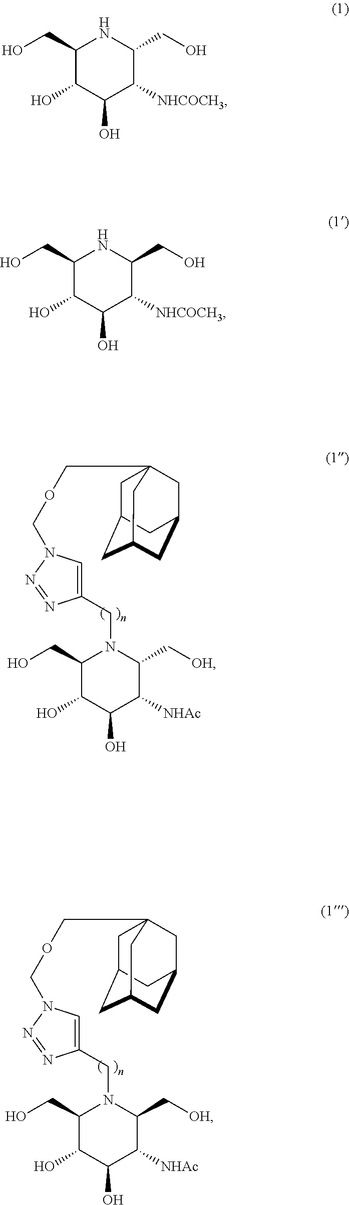 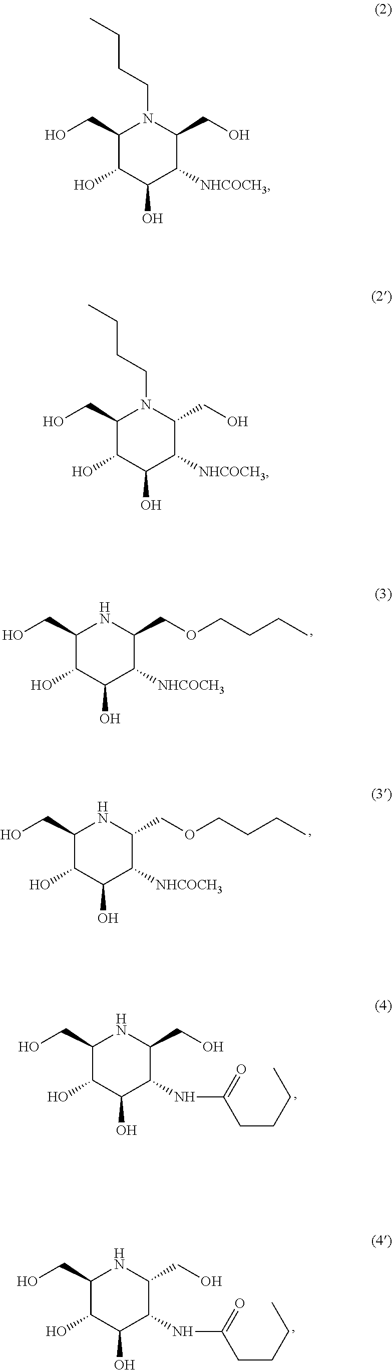 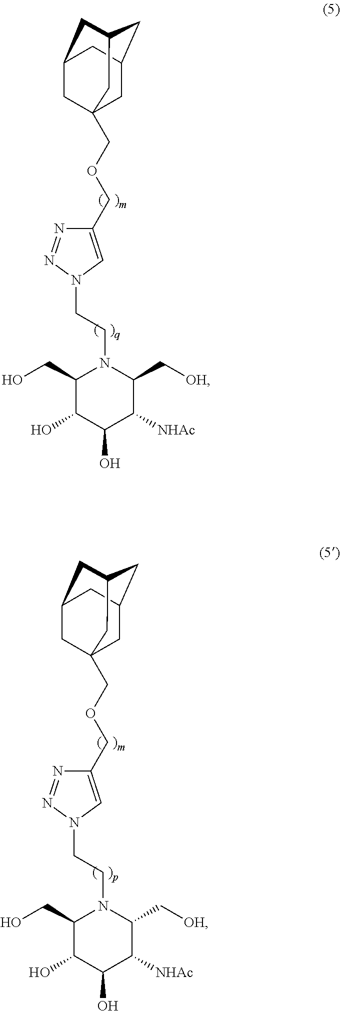 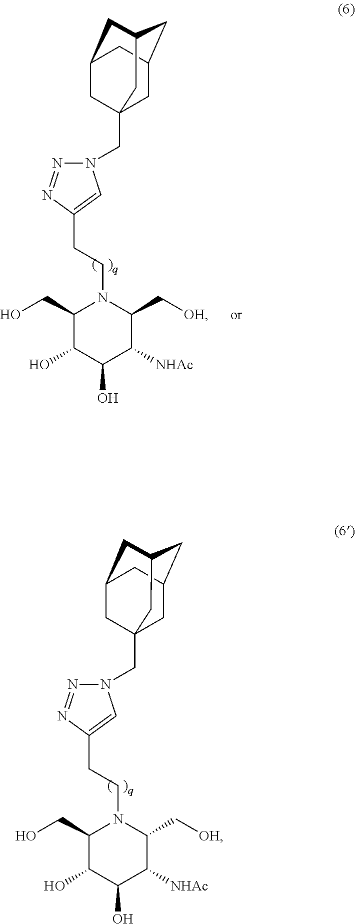
wherein n is an integer from 1 to 10, m is an integer from 1 to 10 and q is an integer from 0 to 9.

8. A compound of general formula (II): 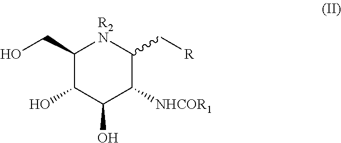
wherein R1 and R2 are as defined in claim 1 and R represents a halogen atom, or a group containing an aliphatic or aromatic group.

9. A compound of the formula (IIIa) or (IIIb): 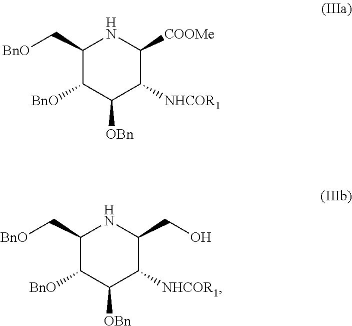
wherein R₁ is as defined in claim 1.

10. A process for preparing a compound as defined in claim 3, comprising the steps of:
(i) hydrolyzing a compound of the following formula (III): 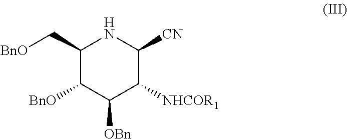
wherein Bn represents an alcohol protecting group, followed by an esterification reaction, as to replace the nitrile function with an ester group,
(ii) reducing the ester group as to obtain an alcohol group, and
(iii) optionally removing the alcohol protecting groups.

11. A process for preparing a compound as defined in claim 4, comprising the steps of:
(i) reacting a compound of the following formula (IV): 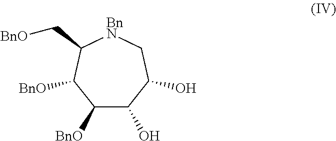
wherein Bn represents an alcohol or amine protecting group, so as to obtain a compound of the following formula (V): 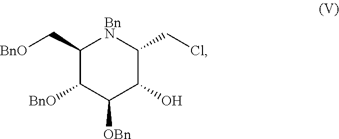 [SMILES: O[C@@H]1[C@@H](OCc2ccccc2)[C@H](OCc2ccccc2)[C@@H](COCc2ccccc2)N(Cc2ccccc2)[C@@H]1CCl]
(ii) preparing from compound of formula (V) a compound of formula (VI): 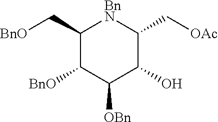 [SMILES: CC(=O)OC[C@@H]1[C@H](O)[C@@H](OCc2ccccc2)[C@H](OCc2ccccc2)[C@@H](COCc2ccccc2)N1Cc1ccccc1]
(iii) preparing from compound of formula (VI) a compound of formula (VII): 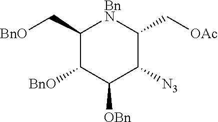 [SMILES: CC(=O)OC[C@@H]1[C@H](N=[N+]=[N-])[C@@H](OCc2ccccc2)[C@H](OCc2ccccc2)[C@@H](COCc2ccccc2)N1Cc1ccccc1]
(iv) preparing from compound of formula (VII) a compound of formula (VIII): 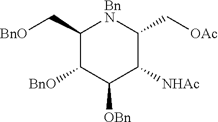 [SMILES: CC(=O)N[C@@H]1[C@@H](OCc2ccccc2)[C@H](OCc2ccccc2)[C@@H](COCc2ccccc2)N(Cc2ccccc2)[C@@H]1COC(C)=O]
(v) preparing from compound of formula (VIII) a compound of formula (IX): 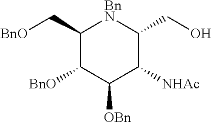 [SMILES: CC(=O)N[C@@H]1[C@@H](OCc2ccccc2)[C@H](OCc2ccccc2)[C@@H](COCc2ccccc2)N(Cc2ccccc2)[C@@H]1CO]
(vi) optionally removing the alcohol and amine protecting groups to obtain compound of formula (1):

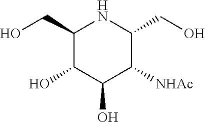 [SMILES: CC(=O)N[C@@H]1[C@@H](O)[C@H](O)[C@@H](CO)N[C@@H]1CO]

12. A pharmaceutical composition comprising one or more compounds as defined in claim 1 and a pharmaceutically acceptable carrier and/or excipient.

13. A method for inhibiting activity of a glycosidase comprising contacting the glycosidase with a compound of claim 1.

14. (canceled)

15. A method for treating a disease selected from the group consisting of type 2 diabetes, neurodegenerative diseases, cancers, viral diseases, and a lysosomal storage disorder, wherein said method comprises administering to a subject in need of such treatment an effective amount of a compound of claim 1.

16. A method for the treatment of dyslipidaemia, haemostasis or fertility control, wherein said method comprises administering to a subject in need of such treatment an effective amount of a compound of claim 1.

17. The compound of claim 5, wherein R2 is a hydrogen atom.

18. The compound of claim 6, wherein R1 is methyl, trifluoromethyl or adamantyl.

19. The compound of claim 8, wherein R is fluoro, alkoxy, halogenoalkoxy, nitrophenoxy, organophosphate, phosphoric acid group, amino acid, peptide, or carbohydrate.

20. The compound of claim 19, wherein alkoxy is methoxy (OMe) or ethoxy (OEt).

21. The compound of claim 19, wherein halogenoalkoxy is —OEtBr.

22. The process of claim 10, wherein the alcohol protecting group is a benzyl group.

23. The process of claim 10, wherein the alcohol protecting groups in step (iii) are removed by hydrogenolysis.

24. The process of claim 11, wherein the alcohol or amine protecting group is a benzyl group.

25. The method of claim 13, wherein the glycosidase is N-acetyl hexosaminidase.

26. The method of claim 15, wherein the neurodegenerative disease is Alzheimer disease.

27. The method of claim 15, wherein the lymosomal storage disorder is selected from the group consisting of Sanfilippo syndrome, Fabry disease, Tay-Sachs disease, and Sandoff disease.
